(19) United States
(12) Patent Application Publication
(10) Pub. No.: US 2024/0182946 A1
(43) Pub. Date: Jun. 6, 2024

(54) METHODS FOR COUNTING THE NUMBER OF LIVING MICROORGANISMS CONTAINED IN A SPECIMEN SAMPLE AND APPARATUSES FOR IMPLEMENTING SUCH METHODS

(71) Applicants: CENTRE NATIONAL DE LA RECHERCHE SCIENTIFIQUE, Paris (FR); UNIVERSITE GRENOBLE ALPES, Saint Martin D'Heres (FR)

(72) Inventors: Bahram HOUCHMANDZADEH, Echirolles (FR); Patrice BALLET, Vernoux en vivarais (FR)

(21) Appl. No.: 18/553,304

(22) PCT Filed: Mar. 25, 2022
(86) PCT No.: PCT/EP2022/058020

§ 371 Date: Sep. 29, 2023

(30) Foreign Application Priority Data
Apr. 1, 2021 (EP) 21305422.4

(51) Int. Cl. C12Q 1/06; C12M 1/42

(52) U.S. Cl. CPC C12Q 1/06; C12M 35/02

(57) ABSTRACT

According to an aspect, the present description is related to an apparatus for depositing on a substrate drops of a specimen sample including a liquid medium and living microorganisms. The apparatus includes a tray for supporting the substrate, a container, e.g. a syringe, configured to receive a volume of the specimen sample and a drop deposition motor configured to push the specimen sample out of the container to form drops of a predetermined volume that detach by gravity and fall on the substrate. The apparatus further includes guiding motors configured for changing a relative position of the tray and the container and a control unit configured to synchronize the drop deposition motor and the guiding motors in order to deposit drops on the substrate according to a pattern.

### PRIOR ART

### Technical field

The present description relates to method for counting the number of living microorganisms contained in a specimen sample. The present description also relates to apparatuses adapted for implementing such methods.

### Prior art

Colony counting, also referred as CFU (“Colony Forming Unit”) is a standard method of counting the number of living microorganisms contained in a liquid sample capable of growth on a specific medium. This very versatile method is the standard technique to measure the number of bacteria for example in water or milk to ensure public safety, in medical samples such as urine or blood to determine the degree of infection and select the antimicrobial agent to use, in microbiology labs to control the outcome of nearly any experiment.

This remarkable method, summarized in FIG. 1A, was invented at the birth of microbiology by R. Koch , R. J. Petri and F. Hesse in 1880's [Ref. 1] and has been used during the last 130 years with practically no modifications. It is based on the isolation of individual colonies of micro-organisms comprised in a microbiological liquid sample 20 to be analysed. The method comprises dispensing a liquid drop 21 of the microbiological liquid sample onto the surface of a growth culture medium 11, hereafter the “plate”, such as agar in a Petri dish 10, followed by the use of a hand-tool 30, called “spreader”, to manually spread the liquid sample across the surface of the medium. Such operation is often referred to as “streaking”. After incubation for an appropriate amount of time, isolated bacteria on the plate that have duplicated form visible colonies 22. The bacterial concentration is then estimated as N/V, where N is the number of colonies and V the volume of liquid sample deposited on the surface of the growth culture medium.

The streaking appears to be highly repetitious and usually conducted in very high volumes in many pathology diagnostic microbiology laboratories, such as in volumes as high as 1,000 to 15,000 plates per day. It is tedious and laborious work that therefore is prone to error and inaccuracies. Further, the liquid drop deposited at the agar surface is small, usually in the order of 100 μL, and can hardly be distinguished from the agar surface. The operator then exerts various streaks in all directions to spread the drop as uniformly as possible on the surface. If the operator does not exert enough care, the result is a sub-optimal spreading with colonies aggregating in some part of the Petri dish. The sub-optimality increases the errors in the estimation of the true number of living bacteria.

While it is relatively easy to spend some time and to be careful when only few plates have to be prepared, it becomes difficult to keep the quality of spreading for hundreds of plates. FIG. 1B shows a typical sub-optimal plating obtained by a manual method on an 85 mm Petry dish 10.

Further, an often-neglected phenomenon is the damage induced to some bacteria by the physical act of spreading [Ref. 2]. It appears that the amount of damage increases with the time it takes to spread the microorganisms, which is often necessary for a good spreading.

For all the reasons stated above, there is a need for either partial or full automation of such CFU counting method.

Presently, there exists one such method known as “spiral plating” developed by Gilchrist and Campbel [Ref. 3] in 1972, and various robotic setups of this technique are commercially available. In this method, a tube dispenses small amount of liquid sample containing microorganisms from a reservoir on a rotating agar plate. The rotation of the dish is coupled to the movement of the arm holding the tube, resulting in a spiral deposition of liquid sample on the agar surface. Such apparatus enables reaching nearly perfect spreads and further enables automatic dilution on a single Petri dish. However, the tube in contact with the agar plate and configured to dispense the liquid sample from the reservoir is a specific equipment that requires a complete disinfection between two uses.

There are also much more sophisticated methods for manipulating 96 pipette tips, aspirating liquid sample from 96 well plates and depositing them on the surface of the agar [Ref. 4]. However, these are very expensive and cumbersome robots.

The present invention provides an original method for colony counting that enables a nearly perfect spread with standard containers such as syringes, with therefore lower disinfection requirements.

### SUMMARY

In what follows, the term “comprise” is synonym of (means the same as) “include” and “contain”, is inclusive and open, and does not exclude other non-recited elements. Moreover, in the present disclosure, when referring to a numerical value, the terms “about” and “substantially” are synonyms of (mean the same as) a range comprised between 80% and 120%, preferably between 90% and 110%, of the numerical value.

According to a first aspect, the present description relates to a method for counting the number of living microorganisms contained in a specimen sample comprising said living microorganisms in a liquid medium, wherein the method comprises:
providing at least a first substrate;
filling a container with a volume of said specimen sample;
pushing the specimen sample out of the container to produce drops of a predetermined volume that detach by gravity and fall on said at least first substrate;
moving the container relatively to said at least first substrate synchronously with pushing the specimen sample out of the container to drop at least a first plurality of said drops on said at least first substrate;
after a predetermined incubation period, determining an initial concentration of living microorganisms in the specimen sample from the colonies of living microorganisms developed on said at least first substrate.

Compared to methods of the prior art, in the method for counting the number of living microorganisms according to the present description, the deposition of the specimen sample on the substate is contactless meaning that no contact is required between the container and the substrate and can be performed using standard containers as syringes. Further, by dropping drops of a predetermined volume of the specimen sample synchronously with the relative displacement of the container and the substrate, a perfect control of the deposition of the specimen sample on the substrate can be achieved.

In the present description, a syringe is understood as a container for a fluid, liquid or gas, and generally comprises a vessel in which the quantity of the fluid is precisely controlled and a needle at an end of the vessel. The fluid in the vessel can be taken in or expelled from by different mechanisms such as the action of a fitted piston or the action of a pump controlling the pressure inside the vessel.

According to one or further embodiments:
the liquid medium of the specimen sample comprises a nourishing medium adapted to develop colonies of living microorganisms from said living microorganisms comprised in the specimen sample;
said at least first substrate is a non-wetting solid substrate so that the drops of said first plurality of drops produce a pattern of at least a first plurality of isolated drops on said at least first substrate; and
said initial concentration of living microorganisms in the specimen sample is determined from a ratio between the number of empty drops in which no colony was developed and the total number of said first plurality isolated drops on said at least first substrate.

In this method, determination of the initial concentration of living microorganisms in the specimen sample is no longer made by determining the number of colonies but is made by determining the number of empty drops, i.e. the drops in which no colony developed. This is made possible by producing isolated drops on the substrate instead of a continuous flow of liquid as in the methods of the prior art. Producing isolated drops is possible by nourishing the specimen sample prior to its deposition on the substrate and using a non-wetting solid substrate, while in the methods according to the prior art, an agar plate is used, which prevents the possibility of depositing isolated drops.

The non-wetting solid substrate may comprise glass or plastic. It may be made for example of a so-called Petri dish but with no growth culture medium such as agar in it.

According to one or further embodiments, a minimal edge-to-edge distance between two drops is determined such that fusion between adjacent drops is prevented. For example, a minimal edge-to-edge distance between two drops is about 1 mm, more advantageously about 2 mm.

According to one or further embodiments, said first plurality of drops comprise a minimum number of around 50 drops, advantageously a minimum number of around 150 drops, to enable a better accuracy in the counting of the empty drops.

In some embodiments, the first plurality of drops may be dropped on a plurality of non-wetting solid substrates, thus enabling a higher number of drops and a better accuracy.

According to one or further embodiments, said pattern is a regular two-dimensional pattern. Such regular two-dimensional pattern enables a better accuracy in the counting of the empty drops. For example, the drops may be located at the nodes of a two-dimensional regular grid. However, other patterns are possible.

According to one or further embodiments, the method further comprises dropping a plurality of drops at a same location of the pattern to increase the volume of said isolated drops of said first plurality of isolated drops, produced on said at least first substrate.

According to one or further embodiments, said first plurality of isolated drops are produced on a first area of said at least first substrate and the method further comprises producing at least a second plurality of isolated drops on a second non-wetting solid substrate or on a different area of said at least first substrate, wherein the isolated drops of the first plurality of isolated drops and the isolated drops of the second plurality of isolated drops have different volumes.

It is thus possible to determine the initial concentration of the living microorganisms in the specimen sample from different pluralities of isolated drops having different volumes. Especially when said initial concentration is not known, such method enables a better accuracy in the estimation of the initial concentration.

According to one or further embodiments, the method further comprises, after incubation, illuminating the substrate with a UV light. The vast majority of microorganisms, such as bacteria, produce fluorescent molecules (process known as “auto-fluorescence”). This property may be used in the method according to the present description to facilitate the counting of the empty drops (or the counting of non-empty drops).

According to one or further developments, the method further comprises, after incubation, illuminating the substrate with a lateral illumination, such as a light ring. When observed from above, drops with bacteria will be seen as light, while drops without bacteria will be seen as dark. Such technique is akin to Dark Field imaging.

According to one or further embodiments, the method further comprises drying the substrate after determining the initial concentration of living microorganisms to preserve the microorganisms. Drying is made during a predetermined drying period, for example a drying period larger than about 2 hours. Microorganisms can be preserved in a natural bio-membrane, which is made possible in the aforementioned method since the substrate is a solid substrate. The micro-organisms can be revived later by addition of liquid medium into the dried trace of at least one or a plurality of drops.

According to one or further embodiments:
said at least first substrate comprises a nourishing medium adapted to develop colonies of living microorganisms from said living microorganisms;
the determining of an initial concentration of living microorganisms in the specimen sample comprises counting the number of colonies developed on said at least first substrate.

In the above method, a traditional agar plate may be used as the substrate. However, due to the contactless deposition of the specimen sample on the substrate by dropping drops of a predetermined volume, a nearly perfect spread can be achieved in a method that doesn't need specific containers and thus have low disinfection requirements.

According to one or further embodiments, the method according to the first aspect further comprises at least a first dilution step of the specimen sample, thus enabling the determination of the concentration of the living microorganisms in at least a second specimen sample whose concentration in living microorganisms differs from that of the initial specimen sample. Such dilution step enables increasing the accuracy of the determination of the concentration of the living microorganisms in the specimen sample, especially when an order of magnitude of the concentration is not known. A plurality of dilution steps may be performed.

For example, according to one or further embodiments:
the container is filled with a first volume of the specimen sample and the volume of the plurality of drops dropped on said first substrate is smaller than said first volume; the method further comprising:
after dropping the plurality of drops on said first substrate, filling the container with a volume of liquid medium to produce a second specimen sample of said first volume with a smaller concentration of living microorganisms;
dropping a plurality of drops of said second specimen sample on a second substrate or on a second area of said first substrate to determine the concentration of living microorganisms in said second specimen sample.

According to a second aspect, the present description relates to an apparatus adapted for implementing methods according to the first aspect.

More particularly, the present description relates to an apparatus for counting the number of living microorganisms contained in a specimen sample comprising said living microorganisms in a liquid medium, the apparatus comprising:
a tray for supporting at least a first substrate;
a container configured to receive a volume of a specimen sample;
a drop deposition motor configured to push the specimen sample out of the container to form drops of a predetermined volume that detach by gravity and fall on said at least first substrate;
at least one guiding motor configured for changing a relative position of the tray and the container;
a control unit configured to synchronize the drop deposition motor and the at least one guiding motor in order to deposit drops on said at least first substrate according to a pattern.

According to one or further embodiments, the container is a syringe and the specimen sample may be pushed out from the container by the action of a piston or by the action of a pump.

According to one or further embodiments, the at least one guiding motor comprises a plurality of guiding motors, configured for changing a relative position of the tray and the container in a plurality of directions. For example, three guiding motors are used for changing a relative position of the tray and the container in the three axes of an orthonormal coordinate system.

According to one or further embodiments, the apparatus further comprises a drop detachment detector configured to detect the detachment of a drop from said container.

For example, the drop detachment detector comprises a light emitting device and a photodetector, wherein the photodetector is configured to detect a variation of a light beam emitted by the light emitting device when a drop detaches.

Using the apparatus of the second aspect, an initial concentration of living microorganisms in the specimen sample can be determined, after a predetermined incubation period, from the colonies of living microorganisms developed on said at least first substrate. Said determination can be made according to any embodiment of the method according to the first aspect.

More particularly, a number of empty drops in which no colony was developed or a number of colonies developed on said at least first substrate can be counted. The counting can be made with the naked eye or using a camera configured to acquire images of the substrate. Said camera may or may not be part of the apparatus.

According to one or further embodiments, the apparatus further comprises:
a camera configured to acquire images of the substrate after a predetermined incubation period;
a processing unit configured to determine from said images, an initial concentration of living microorganisms in the specimen sample from the colonies of living microorganisms developed on said at least first substrate.

### BRIEF DESCRIPTION OF THE DRAWINGS

Other advantages and features of the invention will become apparent on reading the description, which is illustrated by the following figures:

FIGS. 1A, 1B (already described) illustrate the principle of a CFU counting method according to the prior art implementing a manual plating step and an image of a plating obtained by such manual method.

FIG. 2A illustrates steps of a counting method according to the present description.

FIG. 2B illustrates embodiments of a syringe that may be used as a container in the method according to the present description.

FIG. 3 shows two experimental images of plates after the incubation period, with natural light and under UV illumination.

FIGS. 4A and 4B illustrate respectively an estimated value of the mean number of microorganisms per drop and its relative error, as a function of the relative number of empty drops.

FIG. 5 is a schematic of an apparatus adapted for implementing steps of the method according to the present description.

FIG. 6A, 6B illustrate a scheme of another counting method implemented using an apparatus shown in FIG. 5 and an image of a plating obtained by such manual method.

FIGS. 7A to 7C show schemes illustrating dilution steps in methods according to the present description.

### DETAILED DESCRIPTION

In the detailed description which follows, only some embodiments are described in detail in order to ensure clarity of the description, but these examples are not intended to limit the general scope of the principles that emerge from the present description.

The various embodiments and aspects described in the present description may be combined or simplified in multiple ways. In particular, the steps of the various methods may be repeated, reversed, or performed in parallel, unless otherwise specified.

When, in the present description, reference is made to calculating or processing steps for the implementation in particular of method steps, it is understood that each calculating or processing step may be implemented by software, hardware, firmware, microcode or any appropriate combination of these technologies. When software is used, each calculating or processing step may be implemented by computer program instructions or software code. These instructions may be stored in or transmitted to a storage medium that is readable by a computer (or computing unit) and/or be executed by a computer (or computing unit) in order to implement these calculating or processing steps.

In the figures, identical elements are indicated by the same references.

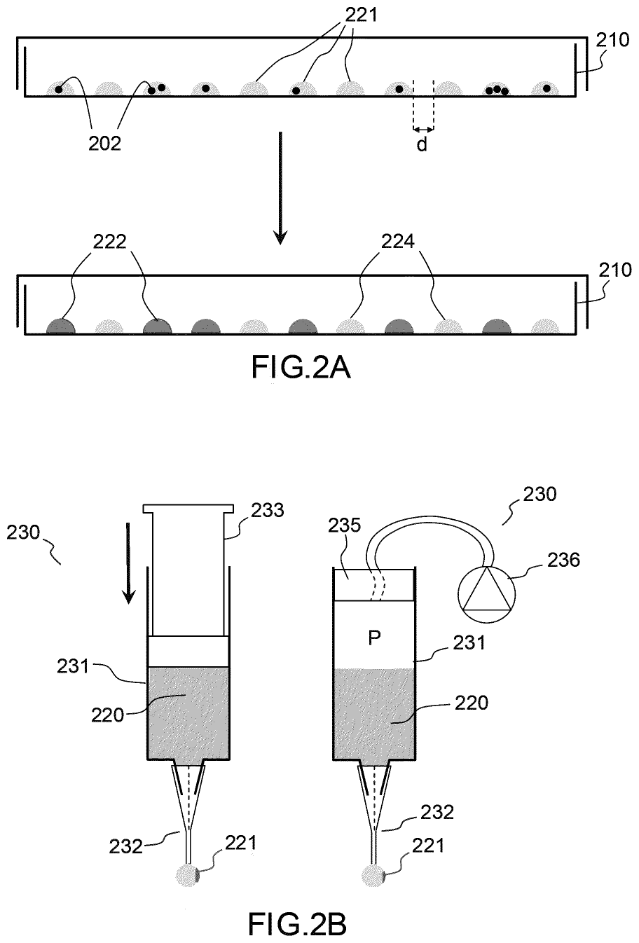

FIG. 2A and 2B illustrate steps of a counting method of living microorganisms contained in a specimen sample according to a first embodiment of the present description.

In the method according to said first embodiment, the specimen sample comprises said living microorganisms in a liquid medium, wherein the liquid medium comprises a nourishing medium adapted to develop colonies of living microorganisms from said living microorganisms.

The method then comprises providing at least a first solid substrate 210, filling a container with a volume of said specimen sample, for example a syringe 230 as it will be described in greater details with reference to FIG. 2B, and pushing the specimen sample out of the container (FIG. 2B) to produce drops 221 of a predetermined volume that detach by gravity and fall on said substrate 210.

The method further comprises moving the container relatively to said at least first substrate synchronously with pushing the specimen sample out of the container to produce at least a first plurality of said drops on said at least first substrate according to a pattern. In the method shown in FIG. 2A, the substrate is a non-wetting solid substrate so that the drops of the first plurality of drops form a pattern of isolated drops 221 on the substrate, i.e. drops that don't fuse.

Although FIG. 2A shows a single substrate, said first plurality of isolated drops mays be produced on a plurality of non-wetting solid substrates, especially if the number of drops is too high for a single substrate.

The method according to said first embodiment then comprises, after a predetermined incubation period, determining an initial concentration of living microorganisms in the specimen sample from the colonies of living microorganisms developed on said at least first substrate. As shown in FIG. 2A, after such incubation period, drops separate in “non-empty drops” 222, in which colonies developed and “empty drops” 224, in which no colony was developed. In the method shown in FIG. 2A, the determining of the initial concentration of living microorganisms in the specimen sample may comprise more specifically counting the number of non-empty drops 222 or the number of empty drops 224 in which no colony was developed, to determine the ratio between the number of empty drops and the total number of isolated drops, as it will be explained in greater details below.

Compared to methods of the prior art, in the methods for counting the number of living microorganisms according to the present description, by dropping drops of a predetermined volume of the specimen sample synchronously with the relative displacement of the container and the substrate, a perfect control of the deposition of the specimen sample on the substrate can be achieved. Further, in the exemplary method of FIG. 2A, producing isolated drops is made possible by nourishing the specimen sample prior to its deposition on the substrate and using a non-wetting solid substrate.

Further, the deposition of the specimen sample on the substate is contactless meaning that no contact is required between the container and the substrate and can be performed using standard containers as syringes.

FIG. 2B shows examples of syringes 230 that may be used in methods according to the present description. The syringe 230 is a container for a fluid, liquid or gas, and generally comprises a vessel 231 with a controlled quantity of fluid 220 and a needle 232 at an end of the vessel. The fluid in the vessel can be taken in or expelled from, by different mechanisms such as the action of a fitted piston 233 (left figure) or the action of a pump 236 controlling the pressure inside the vessel delimited by a fixed plug 235 (right figure). When the liquid is controlled by a piston 233, the movement of the latter controls the level of the liquid in the syringe. When a pump 236 is used, increasing the internal pressure P beyond the atmospheric pressure expels the liquid out of the syringe. Such syringes are standard equipments that can be replaced or disinfected easily.

Determining the initial concentration of living microorganisms in the specimen sample from the number of empty drops 224 is now described in more details.

As previously explained in reference to FIG. 2A, isolated drops 221 are deposited on the non-wetting solid substrate 210, for example the solid surface of a Petri dish, wherein said drops already contain the nourishing media. As the drops fall on a solid surface, their contact angle is strictly superior to 0° so that they remain isolated from one another. The contact angle is for example superior than about 30°, typically comprised between about 60° and about 70°. After the appropriate incubation period, drops 222 that initially contained more than zero microorganism will be filled with a high concentration of bacteria and change their visual aspect. On the other hand, drops 224 that contained no microorganisms will remain the same and not change their aspect.

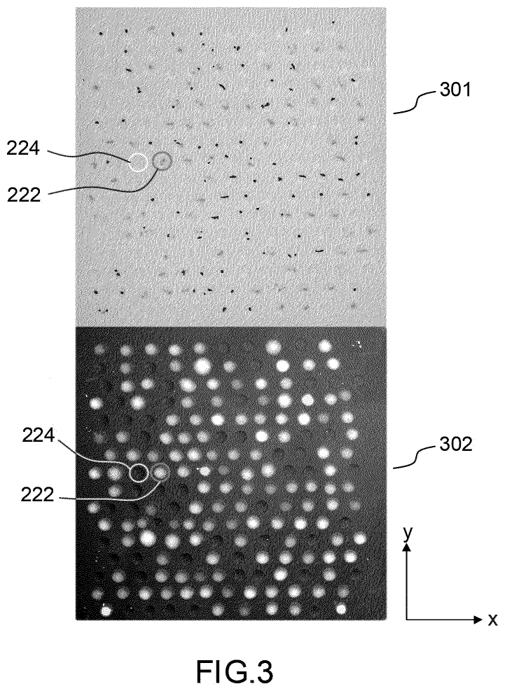

FIG. 3 shows a typical realization of such plating, using a square grid pattern on a 150 mm Petri dish. Distance between two lines is 5.5 mm and images are ≈80×80 cm. Drops can either be visualized by direct observation (301) or by autofluorescence (302) under a standard UV transilluminator with an orange filter.

The method according to said first embodiment of the present description consists of counting the relative number P₀ of unfilled drops after incubation and from this quantity, compute the actual concentration of microorganisms in the liquid. It is thus referred to as “P₀ method” in the present description. The method is akin to transforming the “analog” method of spread counting known in the prior art into a “digital” method of counting zeros and ones.

The mathematical principle of this method is now described.

Consider N isolated drops of a liquid containing microorganisms at concentration C (number/mL) on a solid substrate. Each drop has a volume V. Defining

λ=C×V (1)

as the mean number of organisms per drop, the probability that initially a drop contained n organisms is Poisson distributed:

$${P}_{n} = {e}^{- λ} \frac{{λ}^{n}}{n !} \qquad ( 2 )$$

where n!=1×2× . . . ×n. The probability that a drop contains no organism is therefore

P₀=e^{-λ} (3)

The P₀ method therefore consists of counting the number of drops N₀ that after incubation have stayed the same and determine:

$${P}_{0} = \frac{{N}_{0}}{N} \qquad ( 4 )$$

Relation (3) is then used to estimate directly the mean number of organisms per drop:

λ=−1n(P₀) (5)

where “ln ( )” is the natural logarithm function.

This leads in turn to the value of microorganisms' concentration in the original liquid:

$$C = \frac{λ}{V} = \frac{- ln ( {P}_{0} )}{V} \qquad ( 6 )$$

As explained above, compared to methods of the prior art, the method according to the first embodiment does not necessitate prior preparation of a substrate.

Further, the total amount of liquid deposited on the surface is of order of 1 mL. After the incubation and counting period, the Petri dish can be easily washed for future use, circumventing waste management.

Drops may be deposited on nodes of a regular grid pattern, as shown in FIG. 3. Therefore, only the filled/unfilled (1/0) information is used, making the counting extremely fast and easily automated.

Determining the drops that remain empty after incubation can be achieved by different measurements. One can directly observe the drops that are filled (i.e. non-empty drops) and develop a milky halo or spots inside (FIG. 3, 301). On the other hand, most bacteria produce fluorescent by-products [Ref. 5] that usually are a hindrance for investigators. Here, this hindrance can be put to use to observe drops in which growth has occurred under a standard laboratory transilluminator (FIG. 3, 302). Using fluorescent by-products of bacteria would not be possible in known methods of the prior art due to the fluorescence of agar in traditional Petri dishes. Finally, after the incubation period, Petri dishes can be allowed to dry. Upon drying, filled drops leave very characteristic traces that can be readily identified. The dried Petri dishes can be stored for long periods (>1 month) and living bacteria can be easily retrieved from them if needed. All these methods lead to the same determination of P₀ within an error less than about 1%.

As shown in the non-limitative example of FIG. 3, 65 drops over 195 ones are not filled, leading to P₀=0.29. Drop volumes are V=10.5 μL, leading to C=117.2 Bact/mL.

The error estimate for the P₀ method is now described. Contrary to counting methods according to the prior art, the collisions between colonies and competition for resources has no importance because there are no microorganisms in the unfilled drops 224.

The statistical error can be estimated as follow.

We suppose that from a specimen sample comprising living microorganisms at concentration C, a plurality of N isolated drops of volume V_{d} have been deposited. The value of the mean number of microorganisms per drop (equation 1 above) is λ₀=CV_{d}. The random variable X: “a drop contains at least one microbe” is a binary variable: X=0 with probability p=e^{-λ}^{₀} and X=1 with probability 1−p. This is a Bernoulli random variable with an average value μ=N(1−p) and a standard deviation value σ=\sqrt{p(1−p)}. The standard error σₛ is therefore σₛ=\sqrt{p(1−p)/N}.

From the number N₀ of unfilled drops, we can determine an estimated value pₑ of p and its standard error δ pₑ:

$${p}_{e} = \frac{{N}_{0}}{N} and δ {p}_{e} = \sqrt{{p}_{e} ( 1 - {p}_{e} ) / N} \qquad ( 7 )$$

From this estimation, we can determine an estimated value λₑ of the mean number of microorganisms per drop: λₑ=−ln (pₑ) and its error:

$$λ e + δ λ e = - ln ( {p}_{e} + δ {p}_{e} ) = - ln ( {p}_{e} ) - \frac{δ {p}_{e}}{{p}_{e}} \qquad ( 8 )$$

and therefore, the relative error R in estimation of the mean number of microorganisms per drop is:

$$R = \vert \frac{δ λ e}{λ e} \vert = \frac{δ {p}_{e}}{{p}_{e} ln ( {p}_{e} )} = \frac{1}{\sqrt{N}} \frac{\sqrt{1 - {p}_{e}}}{\sqrt{{p}_{e}} \vert ln ( {p}_{e} ) \vert} \qquad ( 9 )$$

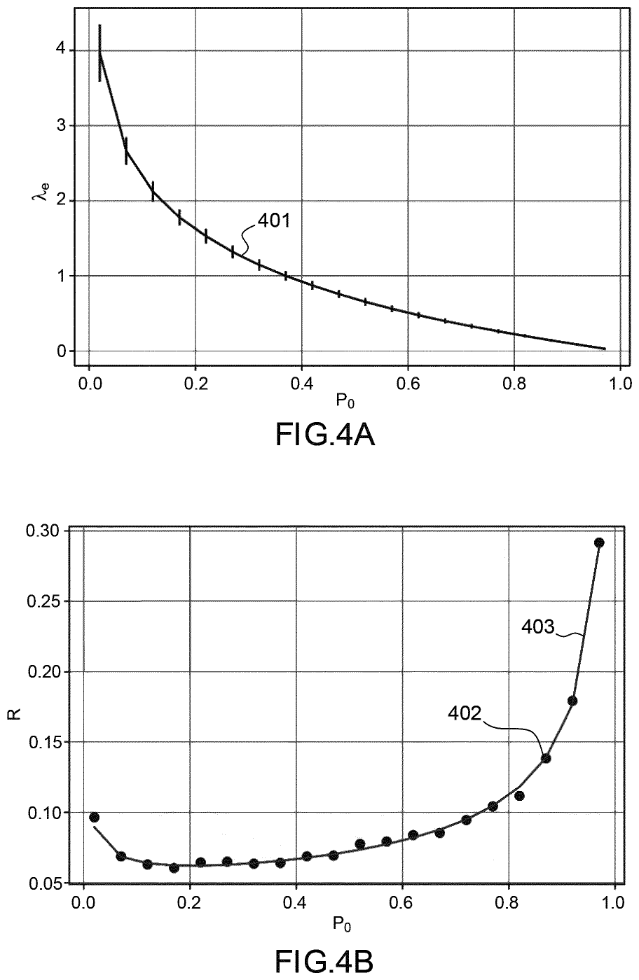

FIGS. 4A, 4B illustrate respectively an estimated value λₑ of the mean number of microorganisms per drop and its relative error R as defined in equation (8) above, as a function of the relative number of empty drops P₀. As a matter of example, for N=400, when the proportion of unfilled drops is 1%, the relative error in estimation of λ is R≈10%. On the other hand, when 80% of drops are empty, R≈11%. The dynamic range of the P₀ method is therefore around 80, about one order of magnitude larger than the spread method.

FIGS. 4A and 4B represent curves showing respectively estimation of mean number λ of bacteria per drop and its relative error R=δλ/λ as a function of the proportion P₀ of counted unfilled drops (equation 9 above). The theory (curve 403) is confirmed by numerical simulations (dots 402). The number of deposited drops in this computation is N=400.

In the example of FIG. 3, drops of equal volume are arranged in the nodes of a two-dimensional regular square. However, drops could be arranged in the nodes of any rectangular grid or hexagonal grid for example.

On the other hand, grids with different drop volumes and/or different spacings could be imprinted on the same or different substrates, allowing the extension of the dynamic range of measurements.

More particularly, providing pluralities of isolated drops of different volumes may be achieved using the method according to said first embodiment by dropping on a first area of the substrate a first plurality of drops of a first predetermined volume and on another area of the substrate, or on a different substrate, a second plurality of drops of a second predetermined volume, for example larger than the first volume. The second predetermined volume is for example obtained by dropping a plurality of drops at a same location of the pattern, thus increasing the volume of the drops produced on the substrate.

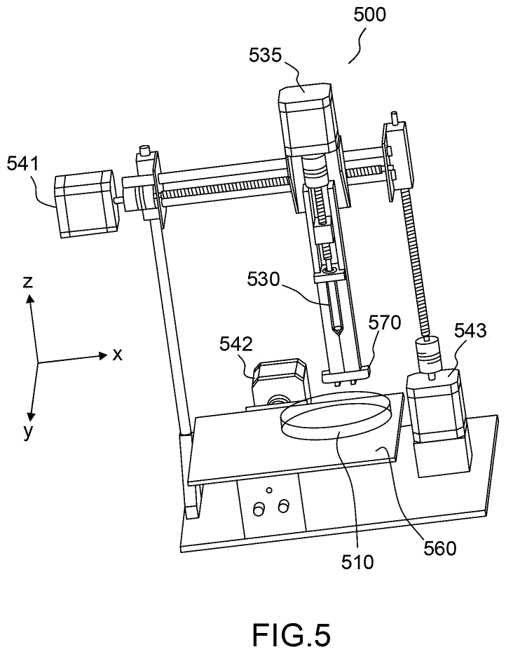

FIG. 5 is a schematic of an apparatus 500 configured for implementing steps of methods according to the present description. In particular, such apparatus is configured for depositing on a substrate 510 drops of a specimen sample comprising a liquid medium and living microorganisms.

As shown in the example of FIG. 5, the apparatus comprises a tray 560 for supporting the substrate 510, a container 530 configured to receive a volume of the specimen sample and a drop deposition motor 535 configured to push the specimen sample out of the container to form drops of a predetermined volume that detach by gravity and fall on the substrate.

The apparatus 500 further comprises at least one guiding motor configured for changing a relative position of the tray and the container. In the exemplary apparatus of FIG. 5, the apparatus 500 comprises the guiding motors 541, 542 and 543 for changing the relative position of the tray and the container respectively in the directions x, y and z, wherein x and y are two perpendicular directions contained in the plane of the tray 560 and z is a direction perpendicular to the plane of the tray 560. The tray 560 is for example mobile in the y direction, while the container is for example mobile in the x direction. Obviously, the tray and/or the container could be mobile in at least one of the directions x, y or z.

The apparatus 500 further comprises a control unit (not shown in FIG. 5) configured to synchronize the drop deposition motor and the at least one guiding motor in order to deposit drops on the substrate according to a pattern. The substrate 510 may be a Petri dish or a glass plate for example. The tray 560 may support the substrate 510 and, optionally, other containers for example for automatic filling of the container 530.

The container 530 is for example a syringe, for example as shown in FIG. 2B, configured for receiving the specimen sample. The drop deposition motor 535 may be a syringe driver as in the example of FIG. 5, or a pump, or any similar device.

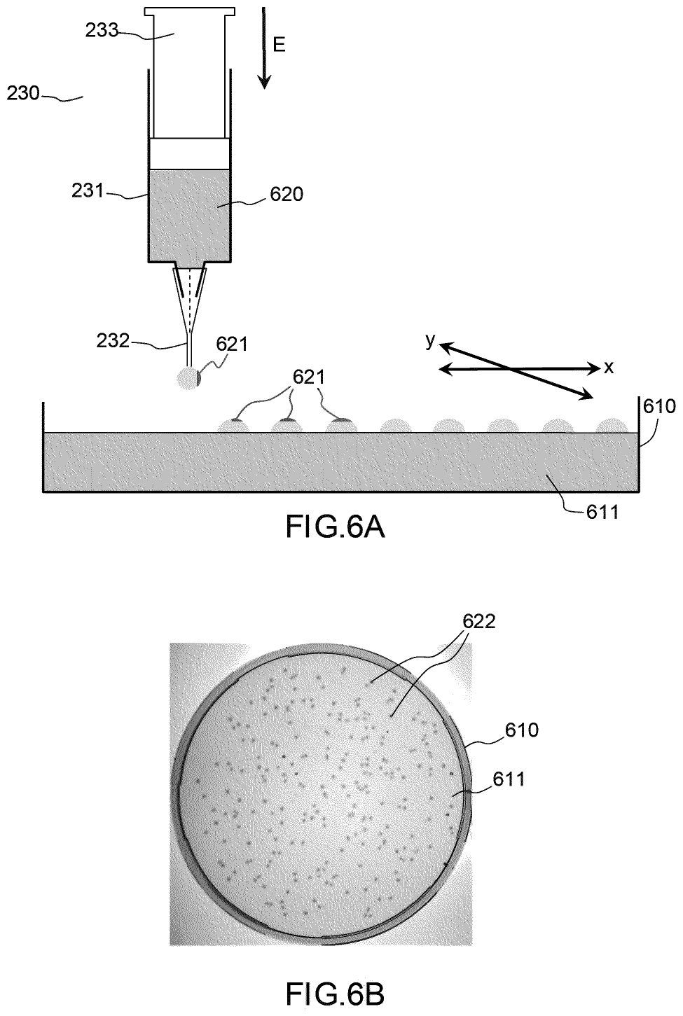

In operation, the substrate 510, e.g. a Petri dish, is positioned on the tray 560 and is moved sequentially in x and y directions relative to the container 530, e.g. a syringe, containing the specimen sample with the living microorganisms. Individual drops are formed by pushing down the specimen sample using the motor 535, configured for either driving a piston, or activating a pump or any similar method. When drops reach a critical volume V, depending on the needle size of the syringe, they detach by gravity and fall on the substrate 510. Synchronizing the speed of xy scan and the motor 535 results in a perfect spread. The substrate may be a neutral one, such as the plastic bottom of the Petri Dish for microbial growth in isolated drops as described in the first embodiment of the method according to the present description. The substrate may be a classic nourishing one such as an appropriate agarose gel, as it will be described in reference with FIG. 6A, 6B, in a method according to a second embodiment of the present description.

The apparatus 500 may further comprise a drop detachment detector 570 configured to count and/or to enhance accuracy of drop positioning on the substrate. The drop detachment detector 570 comprises for example an optical system, with a light emitting device, such as for example a light-emitting diode (LED) and a photodetector facing the light-emitting device. Due to the absorbance of the specimen sample, the photodetector may detect a variation in a light beam emitted by the light emitting device when a drop detaches and passes through the light beam. In some embodiments, the container may be continuously moving in the x direction and the drop detachment detector 570 detects the drop, thus enabling an accurate knowledge of the position of the drop on the substrate. In some other embodiments, the container may be moved only when a drop is detected, thus also enabling a control of the position of the drop on the substrate. Such drop detachment detector may also be an electrical detector, for example an accelerometer or force detector connected to the substrate to detect a change of mass.

The apparatus 500 may further comprise a camera (not shown in FIG. 5) configured to acquire images of the substrate after a predetermined incubation period. The apparatus 500 may further comprise a processing unit configured to determine from said images, an initial concentration of living microorganisms in the specimen sample from the colonies of living microorganisms developed on the substrate. It is thus possible to automate the counting of the concentration of living microorganisms in the specimen sample.

FIG. 6A illustrates a scheme of a counting method according to a second embodiment of the present description. Said method may be implemented using an apparatus as shown in FIG. 5. FIG. 6B illustrates an image of a plating obtained by such method.

In the method according to the second embodiment, the substrate 610 comprises a nourishing medium 611 (such as agarose) adapted to develop colonies of living microorganisms from said living microorganisms. The substrate may be a Petri dish filled with such nourishing medium, as in methods according to the prior art.

As in the method according to the first embodiment, the method according to the second embodiment comprises filling a container with a volume of a specimen sample 620 comprising the living microorganisms in a liquid medium. The container is for example a syringe 230, as illustrated in FIG. 2B. The method then comprises pushing the specimen sample out of the container to produce drops 621 of a predetermined volume that detach by gravity and fall on the substrate 610. The method further comprises moving the container relatively to the substrate synchronously with pushing the specimen out of the container to drop a plurality of said drops on the substrate according to a pattern and, after a predetermined incubation period, determining an initial concentration of living microorganisms in the specimen sample from the colonies of living microorganisms developed on the substrate.

In the method according to the second embodiment, however, since the substrate already comprises a nourishing medium, the drops, when falling on the substrate, spread and fuse as in the counting method according to the prior art. The initial concentration of living microorganisms in the specimen sample is thus determined by counting the number of colonies 622 developed on the substrate after an incubation period, as in the CFU method according to the prior art.

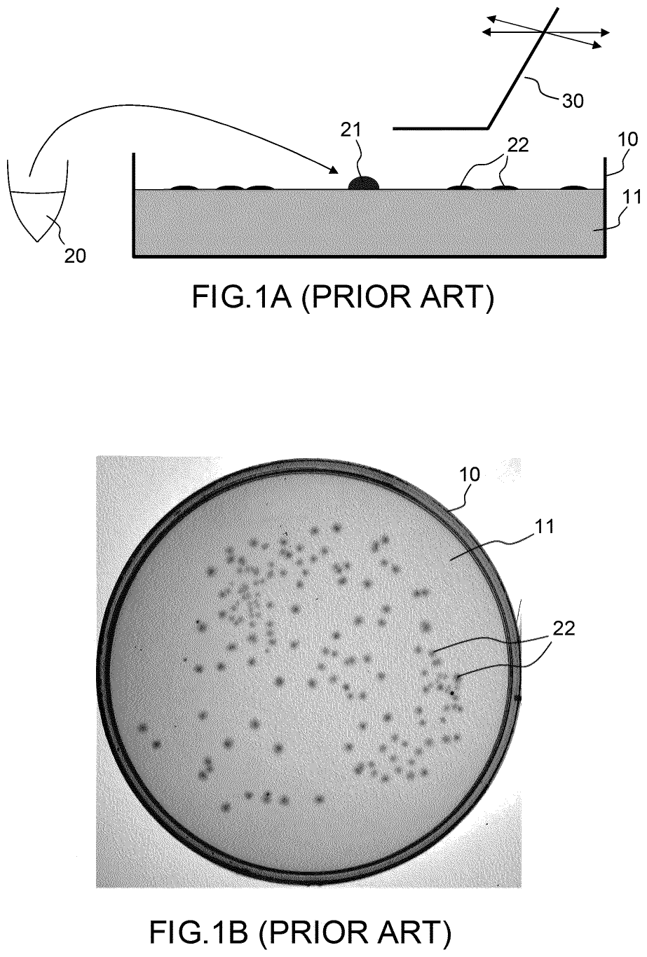

However, and contrary to the manual method according to the prior art and illustrated in FIGS. 1A, 1B, the method according to the second embodiment enables reliable, reproducible and perfect dispersion of microorganisms on the substrate, as shown in FIG. 6B. An example of a plating of bacteria on a 150 mm Petri dish, achieved using an apparatus as shown in FIG. 5 and implementing the method according to the second embodiment is shown in FIG. 6B.

Inventors have shown that the quality of spreading can be evaluated by a “Coefficient of Variation method”, as described in [Ref. 6]. According to this method, the area of the Petri dish is divided into M squares. The numbers nᵢ of colonies in each square are counted and the average μ and variance Vᵣ of these numbers are computed. A perfect spread corresponds to a coefficient of variation C_{V}=Vr /μ≈1, while for a sub-optimal spreading, C_{V}>1. While it is relatively easy to spend some time and to be careful when only few plates have to be made, it becomes difficult to keep the quality of spreading when hundred plates have to be made.

FIG. 1B shows a typical sub-optimal plating obtained by a manual method on a 85 mm Petry dish (C_{V}=3.8 in this example). The quality of the spreading, as measured by the spatial coefficient of variation, is C_{V}=1.1 in the example of FIG. 6B, which is close to the theoretical limit.

Obviously, the apparatus shown in FIG. 5 may be used both to implement methods according to the first embodiment and methods according to the second embodiment, using for example different substrates specifically adapted to each method, positioned on the tray.

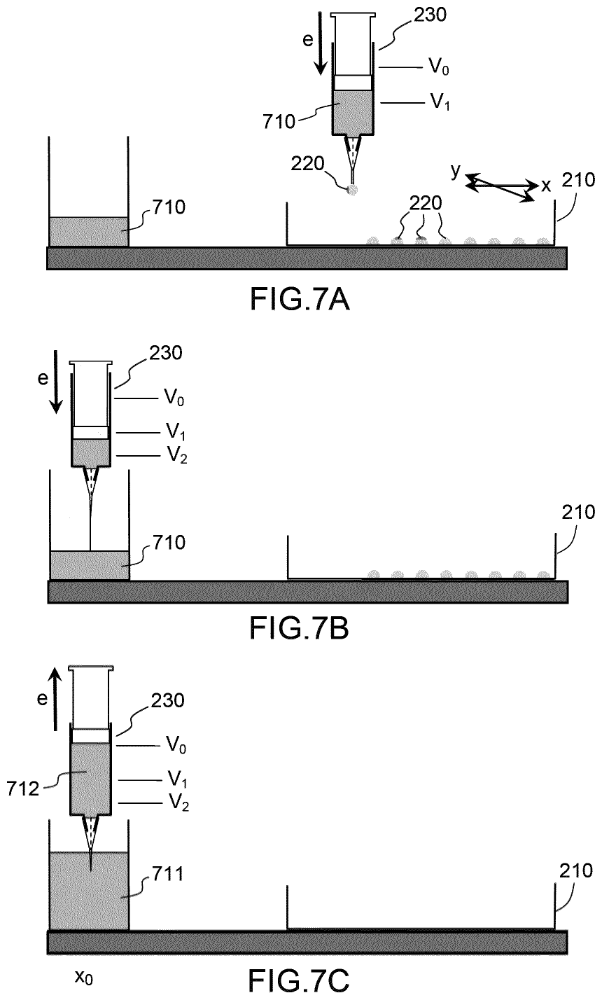

FIG. 7A-7C shows schemes illustrating dilution steps in methods according to the present description. Such dilution steps, although illustrated in a method according to the first embodiment (FIG. 2A, 2B), may be implemented as well in a method according to the second embodiment (FIG. 6A).

In a first step (FIG. 7A), a volume V₀ of the specimen sample containing living microorganisms at concentration C₀ is loaded into the container 230, e.g., a syringe. Isolated drops 220 of a predetermined volume are deposited on the surface of the Petri dish 210 and a total volume Vₜ of the specimen sample is pushed out of the container and. At the end of this step, the container contains a volume V₁=V₀−Vₜ of the original specimen sample.

After the end of the deposition stage shown in FIG. 7A, the container is partially emptied such that only a volume V₂ of the specimen sample remains in the container (FIG. 7B). for example, combining the linear motion of motors x, y, z of the apparatus 500 shown in FIG. 5, the container 230 is moved to the side of the tray at a position x₀, y₀ where an additional volume V_{w} of specimen sample into a waste vessel. At the end of this step, the container 230 contains a volume V₂=V₁−V_{w} of the original fluid.

After the end of the step shown in FIG. 7B, the container is filled with a volume of dilution liquid with no microorganisms in it (FIG. 7C), so that the volume of the new specimen sample in the container is back to V₀. For example, by combining the linear motion of motors y, z, the container 230 is moved to a side of the tray at a position x₀, y₀ where a vessel containing the dilution liquid without microorganisms is positioned. Reversing the direction of motor (e), a volume V_{f}=Vₜ+V_{w} is loaded into the container 230. At the end of this stage, the container 230 contains again a volume V₀ of liquid with microorganisms at concentration C₁, where

C₁=(V₂/V₀)C₀ (10)

Therefore, a dilution factor of (V2/V0) has been achieved. The device is now ready to deposit drops into a new Petri dish. Of course, the steps shown in FIGS. 7A-7C may be reproduced to provide a plurality of dilutions.

Although described by way of a number of detailed example embodiments, the methods and apparatuses according to the present description comprise various variants, modifications and improvements that will be obvious to those skilled in the art, it being understood that these various variants, modifications and improvements fall within the scope of the invention such as defined by the following claims.

### REFERENCES

Ref. 1: R. J. Petri “Eine kleine Modification des Koch'schen Plattenverfahrens”. Centralblatt für Bakteriologie und Parasitenkunde, 1:279-80 (1887).
Ref. 2: P. Thomas et al. “Nonrecovery of varying proportions of viable bacteria during spread plating governed by the extent of spreader usage and proposal for an alternate spotting-spreading approach to maximize the CFU”. J Appl Microbiol Volume: 113. ISSN: 1365-2672 Issue: 2 (2012).
Ref. 3: J. E. Gilchrist et al. “Spiral Plate Method for Bacterial Determination”. J APPLIED MICROBIOLOGY, Vol. 25, No. 2, p. 244-252 (1973).
Ref. 4: R. Hazan et al. “A method for high throughput determination of viable bacteria cell counts in 96-well plates”. BMC Microbiology 12:259 (2012).
Ref. 5: I. Mihalcescu et al. “Green autofluorescence, a double-edged monitoring tool for bacterial growth and activity in microplates”. Physical Biology, 12 (2015).
Ref. 6: B. Houchmandzadeh “Theory of neutral clustering for growing populations”, PHYSICAL REVIEW E 80, 051920 (2009).

Listing of claims:

1. A method for counting the number of living microorganisms contained in a specimen sample comprising said living microorganisms in a liquid medium, wherein the method comprises:
providing at least a first substrate;
filling a container with a volume of said specimen sample;
pushing the specimen sample out of the container to produce drops of a predetermined volume that detach by gravity and fall on said at least first substrate;
moving the container relatively to the substrate synchronously with pushing the specimen out of the container to produce at least a first plurality of said drops on said at least first substrate; and
after a predetermined incubation period, determining an initial concentration (C) of living microorganisms in the specimen sample from the colonies of living microorganisms developed on said at least first substrate.

2. The method according to claim 1, wherein:
the liquid medium comprises a nourishing medium adapted to develop colonies of living microorganisms from said living microorganisms comprised in the specimen sample;
said at least first substrate is a non-wetting solid substrate so that the drops of said first plurality of drops produce a pattern of at least a first plurality of isolated drops on said at least first substrate; and
said initial concentration C of living microorganisms in the specimen sample is determined from a ratio P₀ between the number N₀ of empty drops in which no colony was developed and the total number N of said first plurality of isolated drops on said at least first substrate.

3. The method according to claim 2, wherein a minimal edge-to edge distance (d) between two drops is about 1 mm.

4. The method according to claim 2, wherein said pattern is a regular two-dimensional pattern.

5. The method according to claim 2, further comprising dropping a plurality of drops at a same location of the pattern to increase the volume of said isolated drops of said first plurality of isolated drops produced on said at least first substrate.

6. The method according to claim 2, wherein said first plurality of isolated drops are produced on a first area of said at least first substrate and the method further comprises producing at least a second plurality of isolated drops on a second non-wetting solid substrate or on a different area of said at least first substrate, wherein the isolated drops of the first plurality of isolated drops and the isolated drops of the second plurality of isolated drops have different volumes.

7. The method according to claim 2, further comprising, after incubation, illuminating the substrate with a UV light.

8. The method according to claim 1, wherein:
said at least first substrate comprises a nourishing medium adapted to develop colonies of living microorganisms from said living microorganisms;
the determining of an initial concentration (C) of living microorganisms in the specimen sample comprises counting the number of colonies developed on said at least first substrate.

9. The method according to claim 1, wherein the container is filled with a first volume (V₀) of the specimen sample and the volume (V_{d}) of the plurality of drops dropped on the first substrate is smaller than said first volume; the method further comprising:
after dropping the plurality of drops on the substrate, filling the container with a volume of liquid medium to produce a second specimen sample of said first volume (V₀) with a smaller concentration (C₁) of living microorganisms;
dropping a plurality of drops of said second specimen sample on a second substrate or on a second area of said first substrate to determine the concentration of living microorganisms in said second specimen sample.

10. An apparatus for counting the number of living microorganisms contained in a specimen sample comprising said living microorganisms in a liquid medium, the apparatus comprising:
a tray for supporting at least a first substrate;
a container configured to receive a volume of said specimen sample;
a drop deposition motor configured to push the specimen sample out of the container to form drops of a predetermined volume that detach by gravity and fall on said at least first substrate;
at least one guiding motor configured for changing a relative position of the tray and the container; and
a control unit configured to synchronize the drop deposition motor and the at least one guiding motor in order to deposit drops on said at least first substrate according to a pattern.

11. The apparatus according to claim 10, wherein the container is a syringe.

12. The apparatus according to claim 10, further comprising a drop detachment detector configured to detect the detachment of a drop from said container.

13. The apparatus according to claim 12, wherein the drop detachment detector comprises a light emitting device and a photodetector, wherein the photodetector is configured to detect a variation of a light beam emitted by the light emitting device when a drop detaches.

14. The apparatus according to claim 10, further comprising:
a camera configured to acquire images of the at least first substrate after a predetermined incubation period; and
a processing unit configured to determine from said images, an initial concentration of living microorganisms in the specimen sample from the colonies of living microorganisms developed on the at least first substrate.

15. The method according to claim 2, wherein a minimal edge-to edge distance (d) between two drops is about 2 mm.

16. A method for counting the number of living microorganisms contained in a specimen sample comprising said living microorganisms in a liquid medium, wherein the method comprises:
filling a container with a volume of said specimen sample;
pushing the specimen sample out of the container to produce drops of a predetermined volume that detach by gravity and fall on at least a first substrate;
moving the container relatively to the substrate synchronously with pushing the specimen out of the container to produce at least a first plurality of said drops on said at least first substrate; and
after a predetermined incubation period, determining an initial concentration (C) of living microorganisms in the specimen sample from the colonies of living microorganisms developed on said at least first substrate.

17. The method according to claim 16, wherein:
the liquid medium comprises a nourishing medium adapted to develop colonies of living microorganisms from said living microorganisms comprised in the specimen sample;
said at least first substrate is a non-wetting solid substrate so that the drops of said first plurality of drops produce a pattern of at least a first plurality of isolated drops on said at least first substrate; and
said initial concentration C of living microorganisms in the specimen sample is determined from a ratio P₀ between the number N₀ of empty drops in which no colony was developed and the total number N of said first plurality of isolated drops on said at least first substrate.

18. The method according to claim 17, wherein a minimal edge-to edge distance (d) between two drops is about 1 mm.

19. The method according to claim 17, further comprising:
dropping a plurality of drops at a same location of the pattern to increase the volume of said isolated drops of said first plurality of isolated drops produced on said at least first substrate.

20. The method according to claim 17, wherein said first plurality of isolated drops are produced on a first area of said at least first substrate and the method further comprises producing at least a second plurality of isolated drops on a second non-wetting solid substrate or on a different area of said at least first substrate, wherein the isolated drops of the first and second plurality of isolated drops have different volumes.
